(19) United States
(12) Patent Application Publication
(10) Pub. No.: US 2024/0418732 A1
(43) Pub. Date: Dec. 19, 2024

(54) BIOMARKERS FOR DIAGNOSING TUBERCULOSIS

(71) Applicant: STELLENBOSCH UNIVERSITY, Stellenbosch, Western Cape Province (ZA)

(72) Inventors: Gerhard WALZL, Stellenbosch, Western Cape Province (ZA); Novel Njweipi CHEGOU, Stellenbosch, Western Cape Province (ZA); Regan Shane SOLOMONS, Stellenbosch, Western Cape Province (ZA); Masilo Charles MANYELO, Stellenbosch, Western Cape Province (ZA)

(21) Appl. No.: 18/699,169

(22) PCT Filed: Oct. 5, 2022
(86) PCT No.: PCT/IB2022/059507

§ 371 Date: Apr. 5, 2024

(30) Foreign Application Priority Data
Oct. 6, 2021 (ZA) 2021/07508

(51) Int. Cl. G01N 33/68; G01N 33/543; G01N 33/92

(52) U.S. Cl. CPC G01N 33/6893; G01N 33/54386; G01N 33/6863; G01N 33/92; G01N 2800/26; G01N 2800/60

(57) ABSTRACT

A method of diagnosing tuberculosis (TB) is provided. The method comprising the step of testing a biological sample from the subject for the presence of CC4 and at least one other biomarker selected from the group consisting of CC4b, procalcitonin, CCL1, apolipoprotein-CIII, RANTES and TNF-α. A device, kit and computer-implemented method for diagnosing (and optionally also treating) TB are also provided.

### CROSS-REFERENCE TO RELATED APPLICATIONS

This application claims priority from South African provisional patent application number 2021/07508 filed on 6 Oct. 2021, which is incorporated by reference herein.

### FIELD OF THE INVENTION

Biomarkers for diagnosing tuberculosis are described herein.

### BACKGROUND TO THE INVENTION

Globally, tuberculosis (TB) remains the leading infectious disease killer with 10 million new cases and 1.4 million deaths reported in 2019. Children under the age of 15 years represented 12% of the worldwide TB burden in 2019. Tuberculous meningitis (TBM), the most severe form of TB, mostly affects young children and has an increased risk of death. The true global burden of TBM is not known as many individuals with TBM remain undiagnosed, untreated, and are not reported to the surveillance systems. It is, however, estimated that 2-5% of new cases of TB are TBM. Despite the availability of anti-tuberculosis therapy, TBM results in death in up to 20% of affected children, and severe neurological sequelae in more than half of the survivors. Delay in diagnosis is one of the major concerns in TBM, leading to poor outcomes.

The clinical features seen in TBM may be similar to those in many forms of sub-acute meningoencephalitis, often resulting in diagnostic confusion. Cerebrospinal fluid (CSF) cell count and biochemistry profiles lack sensitivity and are non-specific to use as the primary method of diagnosis. Although clinical criteria have been developed to improve the diagnosis of TBM, performance variability may occur due to atypical clinical features, especially with prevalence of tuberculosis and human immunodeficiency virus (HIV) co-infection. The visualization of acid-fast bacilli by microscopy of the CSF and mycobacterial culture remain the traditional methods for definite diagnosis of TBM. Although microscopy is cheap and rapid, the test is notoriously insensitive (˜10-15%) in routine practice for the diagnosis of TBM. Mycobacterial culture has better sensitivity (˜60-70%) but is too slow (2-6 weeks) to provide results for timely clinical intervention. Furthermore, culture requires a biosafety level 3 laboratory, which is difficult to implement in resource-limited settings.

Recent molecular technologies, particularly nucleic acid amplification tests, have shown improved accuracy and turn-around time for TBM diagnosis, but none of the existing tests is adequate as a stand-alone method for diagnosis of TBM. The Xpert MTB/RIF assay is a rapid molecular test that is based on a real-time polymerase chain reaction (PCR) cartridge system that allows for simultaneous detection of Mycobacterium tuberculosis (M.tb) and rifampicin resistance within 2 hours. A sensitivity of only around 50-60% for the diagnosis of TBM is achieved with the Xpert MTB/RIF test. A re-engineered assay, termed Xpert MTB/RIF Ultra (Xpert Ultra) has shown higher sensitivity than that of either the initial Xpert or culture in the diagnosis of TBM. Consequently, the WHO recommends Xpert Ultra as an alternative to Xpert as the initial test for TBM. Despite improved accuracy, both Xpert MTB/RIF and Xpert Ultra are not adequate to confidently rule out TBM due to imperfect negative predictive value. Other important challenges of Xpert assays are the relatively high operational cost and infrastructural requirements which limit their implementation or wide use in resource-limited settings.

New methods for diagnosis and management of tuberculosis are therefore needed.

### SUMMARY OF THE INVENTION

According to a first embodiment of the invention, there is provided a method of diagnosing tuberculosis (TB) in a subject, the method comprising the step of testing a biological sample from a subject for the presence of CC4 and at least one other capture agent which binds to a biomarker selected from the group consisting of CC4b, procalcitonin, CCL1, apolipoprotein-CIII, RANTES and TNF-α.

The method may include the steps of:
contacting the biological sample with a capture agent which binds to CC4 and at least one other biomarker selected from the group consisting of CC4b, procalcitonin, chemokine (C-C motif) ligand 1 (CCL1), apolipoprotein-CIII, chemokine (C-C motif) ligand 5 (RANTES) and tumour necrosis factor alpha (TNF-α); and
detecting binding of the capture agents to the biomarkers.

The sample may be tested for the presence of CC4 and at least two other biomarkers selected from the group consisting of CC4b, procalcitonin, CCL1, apolipoprotein-CIII, RANTES and TNF-α.

The sample may be tested for the presence of CC4 and at least three other biomarkers selected from the group consisting of CC4b, procalcitonin, CCL1, apolipoprotein-CIII, RANTES and TNF-α.

The method may comprise testing the sample for CC4b, CC4, procalcitonin and CCL1.

The sample may be tested for the presence of CC4, apolipoprotein-CIII, RANTES and TNF-α.

The sample may be a cerebrospinal fluid (CSF), saliva, sputum, blood or urine sample, or may be a pleural or pericardial effusion.

The tuberculosis may be TB meningitis, pleural TB, TB pericarditis, pulmonary TB, TB lymphadenitis, skeletal TB, spinal TB, military TB, genitourinary TB, liver TB, gastrointestinal TB, TB peritonitis or cutaneous TB.

One or more indicators may be provided to indicate when binding of each of the capture agents and biomarkers occurs.

Detection of two or more of the biomarkers in the sample or a measured signal which equates to a level of biomarker in the sample which is higher than a threshold level of the same biomarker may be an indicator of TB.

According to a second embodiment of the invention, there is provided a device for diagnosing TB, the device comprising:
a means for receiving a sample from a subject suspected of having TB;
capture agents for binding CC4 and at least one other biomarker selected from the group consisting of CC4b, procalcitonin, CCL1, apolipoprotein-CIII, RANTES and TNF-α; and
at least one indicator to indicate to a user of the device when the capture agents bind to the biomarkers.

The capture agents may be selected from the group consisting of antibodies, affibodies, ankyrin repeat proteins, armadillo repeat proteins, nucleic acid aptamers, peptides, carbohydrate ligands, synthetic ligands and synthetic polymers. Preferably, the capture agents are antibodies.

The indicator may indicate binding of the capture agent to the biomarker by electrical, electronic, acoustic, optical or mechanical methods.

The device may further include measuring means for measuring the levels of the detected biomarkers.

The device may further include amplifying means for increasing the sensitivity of the detection of the biomarkers.

The device may be a hand-held point-of-care device.

According to a third embodiment of the invention, there is provided a kit for diagnosing TB, the kit comprising one or more of the following:
capture agents for binding CC4 and at least one other biomarker selected from the group consisting of CC4b, procalcitonin, CCL1, apolipoprotein-CIII, RANTES and TNF-α;
means for obtaining or receiving a biological sample from a subject;
a device for diagnosing TB; and/or
instructions, in electronic or paper form, for performing the method as described above.

According to a further embodiment of the invention, there is provided a method of diagnosing a human subject as having TB and treating the subject, the method comprising the steps of:
testing a biological sample from a subject suspected of having TB for the presence of CC4 and at least one other biomarker selected from the group consisting of CC4b, procalcitonin, CCL1, apolipoprotein-CIII, RANTES and TNF-α;
determining whether the subject has TB based on the detection of the biomarkers in the sample; and
administering an effective amount of TB treatment to the subject if the subject is in need thereof.

According to a further embodiment of the invention, there is provided a computer implemented method for diagnosing TB in a subject, the computer performing steps comprising:
receiving inputted subject data comprising values for levels of CC4 and at least one other biomarker selected from the group consisting of CC4b, procalcitonin, CCL1, apolipoprotein-CIII, RANTES and TNF-α in a biological sample from the subject;
comparing these values with predetermined values for the biomarkers;
determining whether the subject has TB; and
displaying information regarding the diagnosis of the subject.

According to a further embodiment of the invention, there is provided the use of capture agents for binding CC4 and at least one other biomarker selected from the group consisting of CC4b, procalcitonin, CCL1, apolipoprotein-CIII, RANTES and TNF-α in the manufacture of a kit for diagnosing TB.

### BRIEF DESCRIPTION OF THE FIGURES

FIG. 1: STARD diagram depicting the study design and classification of study participants. CRF: case report form; CSF: cerebrospinal fluid; ELISA: enzyme-linked immunosorbent assay; TBM: Tuberculous meningitis; no-TBM: children presenting with signs and symptoms suggestive of TBM, but finally diagnosed with other meningitis or no meningitis (see Table 2).

FIG. 2: Scatter plots showing the concentrations of biomarkers in CSF samples from children with TBM and no-TBM, and the receiver operator characteristics curves showing the accuracies of the biomarkers in the diagnosis of TBM. The error bars depict the median concentrations and interquartile ranges. Unadjusted Mann-Whitney U test p-values for the differences between the medians of the groups are shown for each biomarker. Representative plots for the six most accurate individual biomarkers (AUC>0.90) are shown.

FIG. 3: Performance of CC4b+CC4+Procalcitonin+CCL1 in the diagnosis of TBM in children. Receiver operator characteristic (ROC) curve showing the accuracy of a four-marker CSF biosignature comprising CC4b+CC4+Procalcitonin+CCL1 in diagnosing TBM (A), Scatter plot showing the ability of the biosignature in discriminating between TBM and no-TBM (B), Frequency of proteins in the top 20 general discriminant analysis (GDA) models that accurately classified children with TBM and no-TBM (C).

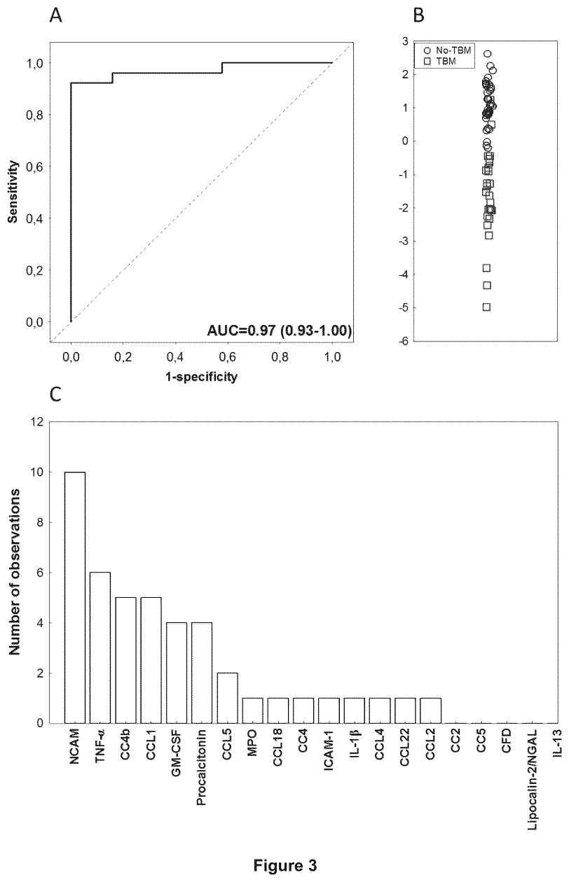

FIG. 4: Performance of Apolipoprotein-CIII+CC4+RANTES+TNF-α in the diagnosis of TBM in children. Receiver operator characteristic (ROC) curve showing the accuracy of a 4-marker CSF biosignature comprising Apo-CIII+CC4+RANTES+TNF-α in the diagnosis of TBM (A), Scatter plot showing the ability of the biosignature in discriminating between TBM and no-TBM (B).

### DETAILED DESCRIPTION OF THE INVENTION

A method, device, kit and computer-implemented method for diagnosing (and optionally also treating) tuberculosis (TB) are described herein.

Abbreviations of biomarkers referred to herein: TGF-α=Transforming growth factor alpha, VEGF-A=Vascular endothelial growth factor A, PDGF AB/BB=Platelet-derived growth factor AB/AA, CCL5 (RANTES)=Chemokine (C-C motif) ligand 5 also known as Regulated upon activation, normally T-expressed, and presumably secreted (RANTES), CD56 (NCAM)=Cluster of differentiation 56 also known as Neural cell adhesion molecule-1, sICAM-1=Soluble intercellular adhesion molecule also known as cluster of differentiation 54 (CD54), MPO=Myeloperoxidase, PDGF-AA, sVCAM-1=Soluble vascular cell adhesion molecule, PAI-1=Plasminogen activator inhibitor-1, CRP=C-reactive protein, SAP=Serum amyloid P, PEDF=Pigment epithelium-derived factor, CCL18 (MIP-4)=Chemokine (C-C motif) ligand 18, also known as Macrophage inflammatory protein 4, AB42=Amyloid beta 42, sRAGE=Soluble receptor of advanced glycation end-products, GDF-15=Growth differentiation factor-15, SAA=Serum amyloid A, CCL1 (1-309)=Chemokine (C-C motif) ligand 1, also abbreviated as I-309, CXCL11 (I-TAC)=C-X-C motif chemokine ligand 11, also known as interferon-inducible T-cell alpha chemoattractant (I-TAC), IFN-γ=Interferon gamma, TNF-α=Tumour necrosis factor alpha, CCL2 (MCP-1)=Chemokine (C-C motif) ligand 2 also known as monocyte chemoattractant protein-(MCP-) 1, CXCL9 (MIG)=C-X-C motif chemokine ligand 9, also known as monokine induced by interferon Gamma (MIG), GM-CSF=Granulocyte-macrophage colony-stimulating factor, IL-1ra=interleukin 1 receptor antagonist, CCL11=Chemokine (C-C motif) ligand 11, also known as Eotaxin, G-CSF=Granulocyte colony-stimulating factor, S100A9=S100 calcium-binding protein A9, FAP=Fibroblast activation protein, CCL4 (MIP-1β)=Chemokine (C-C motif) ligand 4, also known as macrophage inflammatory protein-beta (MIP-1β), CCL3 (MIP-1β)=Chemokine (C-C motif) ligand 3, also known as macrophage inflammatory protein-alpha (MIP-1β), CXCL10 (IP-10)=C-X-C motif chemokine ligand 10, also known as interferon gamma inducible protein 10 (IP-10), CCL22 (MDC)=C-C motif chemokine 22 also known as Macrophage-derived chemokine, CD40 Ligand=Cluster of differentiation 40 ligand.

The method comprises testing a biological sample from a subject for at least one biomarker selected from the group consisting of CC4, CC4b, procalcitonin, CCL1, apolipoprotein-CIII, RANTES or TNF-α.

Typically, (a) the biological sample is contacted with capture agents which can bind to the biomarker(s) of interest, and (b) binding of the capture agents to the biomarker(s) is detected.

The subject may be suspected as having TB or may have been exposed to a patient with a Mycobacterium tuberculosis infection.

The at least one biomarker can be CC4. The at least one biomarker can be CC4b. The at least one biomarker can be procalcitonin. The at least one biomarker can be CCL1. The at least one biomarker can be apolipoprotein-CIII. The at least one biomarker can be RANTES. The at least one biomarker can be TNF-α.

The second biomarker can be CC4b. The second biomarker can be CC4. The second biomarker can be procalcitonin. The second biomarker can be CCL1. The second biomarker can be apolipoprotein-CIII. The second biomarker can be RANTES. The second biomarker can be TNF-α.

The method can further comprise testing the sample for a third biomarker. The third biomarker can be CC4b. The third biomarker can be CC4. The third biomarker can be procalcitonin. The third biomarker can be CCL1. The third biomarker can be apolipoprotein-CIII. The third biomarker can be RANTES. The third biomarker can be TNF-α.

The method can further comprise testing the sample for a fourth biomarker. The fourth biomarker can be CC4b. The fourth biomarker can be CC4. The fourth biomarker can be procalcitonin. The fourth biomarker can be CCL1. The fourth biomarker can be apolipoprotein-CIII. The fourth biomarker can be RANTES. The fourth biomarker can be TNF-α.

In one embodiment, the method comprises testing the sample for CC4, CC4b, procalcitonin and CCL1.

In another embodiment, the method comprises testing the sample for CC4, apolipoprotein-CIII, RANTES and TNF-α.

It will, of course, be possible to test the sample for additional biomarkers, such as one or more of those listed in Table 3.

A positive diagnosis for TB can be made when binding of the capture agents to one, two, three, four or more of the tested biomarkers is detected, or when the levels of the detected biomarkers are higher than a typical level of the same biomarker in subjects without TB. In another embodiment, a positive TB diagnosis can be made when the levels of the detected biomarkers are lower than a typical level of the same biomarker in subjects without TB.

Cut-off or threshold values can be determined based on levels of biomarkers which are typically found in patients without TB, and the levels of the biomarkers detected in the sample can be compared to the cut-off levels when making the determination of whether or not the subject has TB. In other words, the method will detect whether the biomarkers in the panels are under- or over-expressed relative to a subject who does not have TB.

In one embodiment, the method is for diagnosing TB meningitis (TBM). However, the method may also be used for diagnosing pleural TB, TB pericarditis, pulmonary TB, TB lymphadenitis, skeletal TB, spinal TB, military TB, genitourinary TB, liver TB, gastrointestinal TB, TB peritonitis or cutaneous TB. Preferably, the method is for diagnosing active TB (signs and symptoms of TB and/or consistent imaging evidence together with microbiological confirmation (culture positivity and/or presence of amplified DNA)), rather than for diagnosing latent M. tuberculosis infection (LTBI) or incipient TB. The method is independent of HIV co-infection status.

In one embodiment, the sample is a cerebrospinal fluid (CSF) sample. Although a CSF-based test requires an invasive sample collection through lumbar puncture by experienced personnel, the benefits are quick turn-around time of such a test relative to current tests, and thus timely management of TBM.

In other embodiments, the sample can be a blood, saliva, sputum or urine sample or a pleural or pericardial effusion.

The sample can be centrifuged before it is tested. Alternatively, the sample can be tested without centrifugation.

The methods described above can be used to diagnose TB, and in particular TBM, in all human subjects, including adults and children (e.g. children 13 years and younger).

TB treatment can be administered to subjects who are diagnosed as having TB.

The method can also be used as an initial diagnostic tool whereby a positive diagnosis from this method can, if necessary, be subsequently confirmed by means of a second diagnostic method. In the interim, while waiting for the results of the second test, the subject can be started on treatment. Conversely, the method of the invention can also be used to rule out TB, thus preventing overtreatment of non-TB subjects.

The biomarkers can be detected using commercially available techniques, such as ELISA techniques or multiplex bead array technology, although it is intended that a specific point-of-care (or bedside) diagnostic device will be used for rapid diagnosis, particularly in resource poor settings. Such a device will lead to a significant reduction in the costs and delays that are currently incurred in the diagnosis of TB, with a consequent reduction in death or long-term disability.

In one embodiment, the device has a means for receiving the sample from the subject, such as a loading or receiving area onto or into which the sample is placed. Capture agents and indicators are present in the device, and once the sample has been loaded onto or received into the device, the sample is brought into contact with the capture agents, which are allowed to bind to the biomarkers if present. The indicator will indicate to the user of the device that binding of capture agents to one or more of the biomarkers has occurred. The device may further include amplifying means for increasing the sensitivity of the detection of the biomarkers.

The capture agents can be antibodies, affibodies, ankyrin repeat proteins, armadillo repeat proteins, nucleic acid aptamers, peptides, carbohydrate ligands, synthetic ligands or synthetic polymers. Typically, however, the capture agents are antibodies. The indicator can be a calorimetric, electrical, electrochemical, electronic, chromogenic, optical, fluorescent or a radio-labeled indicator.

For example, the point-of-care device can be a lateral flow device similar to those known in the art. This can be dipped into the sample, or the sample can be placed onto a portion of the device commonly known as the sample pad. Fluid from the sample migrates to a portion of the device containing the capture agents, which generate a signal when they bind to the biomarkers in the panel. The device may use up-converting phosphor technology.

Another example of a suitable point-of-care assay makes use of biosensors comprising a transducer element, for the conversion of the biological signal to an electronic signal, to which antibodies against the biomarkers can be immobilised. The transducer element can use different conversion mechanisms, such as piezoelectricity or impedance changes, and can be implemented on different substrates, such as electrospun nanofiber meshes or paper. Depending on the chosen transducer element, the binding of the target molecules in the samples to the immobilised capture antibodies results in the generation of piezoelectric energy or a change in impedance, proportional to the amount of target molecule detected in the sample. The measured data are stored in the handheld device containing the biosensing elements, but can also be downloaded to a database or cloud for further analysis.

A kit can also be provided to enable the method of the invention to be performed. The kit could include one or more of the following:
capture agents, such as antibodies, for binding the intended biomarkers;
a means for obtaining or receiving the sample from a subject;
a point-of-care device as described above; and/or
instructions, in electronic or paper form, for performing the method.

The invention further provides a computer implemented method for diagnosing TB in a subject, the computer performing steps comprising:
a) receiving inputted subject data comprising values for levels of the biomarkers of interest in a sample from the subject;
b) comparing these values with predetermined values for the biomarkers;
c) determining whether the subject has TB; and
d) displaying information regarding the diagnosis of the subject.

The subject may be diagnosed with TB if one or more of the biomarkers is detected in the sample, or if the measured levels of the biomarkers in the sample are higher than a predetermined value for the biomarkers. The predetermined value is generally based on typical levels of the same biomarker in subjects without TB.

The invention further provides the use of capture agents for at least two biomarkers, at least one of which is selected from the group consisting of CC4b, CC4, procalcitonin, CCL1, apolipoprotein-CIII, RANTES and TNF-α in the manufacture of a kit or device for diagnosing TB. For example, this could be a combination of capture agents which bind CC4b, CC4, procalcitonin and CCL1 or a combination of capture agents which bind CC4, apolipoprotein-CIII, RANTES and TNF-α.

The invention will now be described in more detail by way of the following non-limiting examples.

### EXAMPLES

### Study Setting and Participants

Children aged between 3 months and 13 years suspected of having meningitis were recruited at Tygerberg Hospital in Cape Town, South Africa. Written informed consent was provided by parents or legal guardians/caregivers of the participants. Assent was obtained if children were older than 7 years, provided that they had a normal level of consciousness based on the Glasgow Coma Score (GCS), wherein a GCS of 15/15 was considered as normal. Some of the severely ill children admitted to the hospital during the study period were not recruited owing to these children being too ill and requiring emergency treatment. All the study participants were TB treatment naïve at the time of enrolment.

### Procedures

Comprehensive clinical investigation including assessment of the signs and symptoms, history of TB contact, HIV test, GCS, tuberculin skin test (TST), and chest radiography were carried out on all study participants. Routine computed Tomography (CT) of the brain was performed in all suspected TBM cases, and in other forms of meningitis when indicated. Magnetic resonance imaging (MRI) was performed when clinically indicated. Air-encephalography was performed in all TBM cases with hydrocephalus demonstrated on neuroimaging, provided there were no contra-indications to performing a lumbar puncture (LP).

After collection of a lumbar CSF specimen, routine CSF diagnostic investigations included color and appearance, differential cell count determination, protein concentration and glucose levels. Each CSF sample was further microbiologically examined by gram staining, India ink staining, culture of the centrifuged sediment on blood agar plates for pyogenic bacteria, Auramine “O” staining & fluorescence microscopy, culture using the mycobacterium growth indicator tubes (MGIT)™ method (Becton and Dickinson), GeneXpert MTB/RIF and HAIN Genotype MTBDRplus kit for M.tb DNA when the mycobacterial culture was positive. Other additional investigations included viral PCR and the determination of serum glucose levels. Following the collection of specimens for routine diagnostic purposes, additional CSF samples were collected and transported to the research laboratory for this study. The CSF samples were centrifuged at 4000×g for 15 minutes, after which the supernatants were harvested and stored at −80° C. until the protein biomarkers were measured.

### Classification of Study Participants

Definite TBM, probable TBM, and no-TBM were defined. Briefly, (1) Children with microbiologically-confirmed TBM by either the detection of acid-fast bacilli in the CSF, positive CSF culture or a positive CSF GeneXpert MTB/RIF result were classified as having definite TBM; (2) Probable TBM cases were defined based on a scoring system that combines clinical criteria, CSF criteria, neuroimaging criteria, and evidence of TB elsewhere; (3) children with alternative diagnoses including bacterial meningitis, viral meningitis, and no meningitis were defined as ‘no-TBM’.

### Immunoassays

The concentrations of 67 host protein biomarkers were measured in CSF samples from all the study participants using either ELISA (transthyretin) or the Luminex multiplex immunoassay platform (all other biomarkers).

CSF transthyretin concentrations were measured using a commercially available kit (Novus Biologicals, Biotechne, USA, Catalog #NBP2-60516) according to the manufacturer's instructions. The absorbance at 450 nm was read using an automated microplate reader (iMark™ Microplate Reader, Bio-Rad Laboratories), and the generated standard curve was used to determine the concentrations in each sample.

The concentrations of the other 66 host protein biomarkers were determined using Luminex multiplex kits supplied by Merck Millipore (Billerica, MA, USA) and R&D systems (Bio-Techne, Minneapolis, USA), according to the manufacturer's instructions (Table 1). Luminex plates were read on either a Bio-Plex 200 or Magpix instrument (Bio-Rad Laboratories), in an ISO 15189 accredited laboratory. Bead acquisition and analysis of median fluorescence intensity were done using the Bio-Plex Manager 6.1 software (Bio-Rad Laboratories). The laboratory staff performing the experiments were blinded to the clinical classification of the children. The quality control samples included in the assays yielded values that were within the expected ranges.

TABLE 1
Reagent kits and analytes evaluated in the current study.

| | |
|---|---|
| Panel name and | |
| catalogue number | Analytes |

Reagent kits and analytes purchased from Merck Millipore, Billerica, MA, USA

| | |
|---|---|
| Human | Interleukin (IL)-13, TGF-α, VEGF-A |
| Cytokine/Chemokine/Growth | |
| Factor Panel A | |
| (HCYTA-60K-03) | |
| Human Neurodegenerative | PDGF-AB/BB, CCL5 (RANTES), CD56 (NCAM), |
| Disease Magnetic Bead Panel 3 | ICAM-1, MPO, PDGF-AA, VCAM-1, PAI-1 |
| (HNDG3MAG-36K-08) | |
| Human Neurodegenerative | CRP, α1-Antitrypsin, SAP, PEDF, CCL18 (MIP-4), |
| Disease Magnetic Bead Panel 2 | Complement Component (CC) 4 |
| (HNDG2MAG-36K-06) | |
| Human Neurodegenerative | Apolipoprotein (APO) AI, Complement Factor H, |
| Disease Magnetic Bead Panel 1 | APO-CIII, CC3 |
| (HNDG1MAG-36K-04) | |
| Human Neurodegenerative | Aβ42, sRAGE |
| Disease Magnetic Bead Panel | |
| (HNDG4MAG-36K-02) | |
| Human Cardiovascular Disease | D-DIMER, GDF-15, sP-SELECTIN, lipocalin-2/NGAL, |
| Magnetic Bead Panel 2 | SAA |
| (HCVD2MAG-67K-05) | |
| Human Complement Panel 1 | CC2, CC4b, CC5, CC5a, Complement Factor D, |
| (HCMP1MAG-19K-07) | Mannose-Binding Lectin (MBL), Complement Factor I |

Reagent kits and analytes purchased from R&D Systems, Minneapolis, MN, USA

| | |
|---|---|
| Human Magnetic Luminex | CD40 Ligand, CCL1 (I-309), IL-1β, IL-12/23p40, CXCL11 |
| Screening Assay 28-plex | (I-TAC), IL-6, CCL4 (MIP-1β), Procalcitonin, CXCL10 |
| (LXSAHM-28) | (IP-10), IFN-γ, IL-10, CCL3 (MIP-1α), CCL22 (MDC), |
| | Matrix metalloproteinase (MMP)-1, TNF-α, CCL2 |
| | (MCP-1), CXCL9 (MIG), GM-CSF, IL-1α, IL-1ra, IL-17, |
| | CCL11 (Eotaxin), G-CSF, IL-4, CXCL8 (IL-8), IL-18, |
| | MMP-8, S100A9 |
| Human Magnetic Luminex | Ferritin, MMP-9, FAP |
| Screening Assay 3-Plex | |
| (LXSAHM-03) | |
| Human Magnetic Luminex | VEGF-A |
| Screening Assay 1-Plex | |
| (LXSAHM-01) | |

### Statistical Analysis

Analyses were performed using Statistica (TIBCO Software Inc., CA, USA) and GraphPad Prism version 9 (Graphpad Software Inc., CA, USA). Differences in the concentrations of single biomarkers were compared between the clinical groups (TBM and no-TBM) using the Mann-Whitney U test. P-values<0.05 were considered significant. The diagnostic abilities of individual biomarkers were evaluated using receiver operator characteristic (ROC) curve analysis. Sensitivity and specificity values were determined by a selection of optimal cut-off values based on Youden's index (Fluss et al., Biom J Biom Z., 2005, 47 (4): 458-72). The diagnostic accuracies of combinations between biomarkers were assessed by general discriminant analysis (GDA), followed by leave-one-out cross-validation. Variable selection for the GDA was done using the all subset regression method. V-fold cross validation was used for selecting best models. Consistency of markers to be selected was evaluated by counting how many times they appeared in the top 20 models. Association between the different biomarkers was analysed by use of Spearman correlation. Assessment of the factor structure of the biomarkers for the total samples was carried utilizing exploratory factor analysis (EFA) with oblimin rotation.

### Results

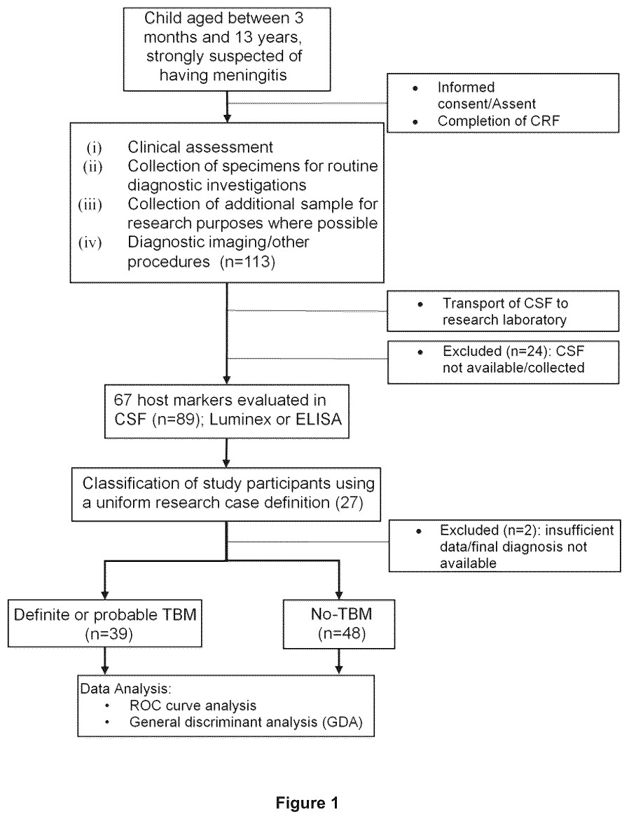

113 children were prospectively enrolled in the study, 87 of whom provided sufficient CSF samples, and were included in the present study. Of these 87 children, 47 (54.0%) were males (FIG. 1). Among 56 with available HIV infection results, 7 (12.5%) were positive. Thirty-nine (44.8%) of the 87 children were diagnosed with TBM (4 definite TBM and 35 probable TBM) and 48 (55.2%) had alternative diagnoses including viral meningitis (n=2), bacterial meningitis (n=5), and no meningitis (n=41). The 48 participants with alternative diagnoses were classified as no-TBM during data analysis. The baseline characteristics of the study participants are shown in Table 2.

TABLE 2
Clinical and demographic characteristics of study participants.

| | All, No (%) | | TBM, No (%) | | No-TBM^{#}, No (%) | |
|---|---|---|---|---|---|---|
| Number of participants | 87 | | 39 | | 48 | |
| Age, median months (IQR) | 24.0 | (11.0-61.0) | 24.0 | (12.0-48.0) | 23.5 | (12.0-85.0) |
| Males, n (%) | 47 | (54.0) | 20 | (51.3) | 27 | (56.3) |
| HIV infection status | 8/61 | (13.1) | 1/33 | (3.0) | 7/28 | (25.) |
| (n/n tested) | | | | | | |
| BCG* done, n (%) | 52 | (59.8) | 26 | (66.7) | 26 | (54.2) |
| TB contact in history | 27 | (31.0) | 18 | (46.2) | 9 | (18.8) |

*Although Bacillus Calmette-Guérin (BCG) vaccination is routinely administered at birth in the study community, BCG vaccination was documented in only 59.8% of the study participants.
^{#}No-TBM group included children diagnosed with viral meningitis (n = 3), bacterial meningitis (n = 4) and no meningitis (n = 41, including Acute flaccid paralysis, Autoimmune encephalitis, cerebral infarction- right middle cerebral artery, Chicken Pox, Complex febrile seizure, Developmental regression, Dural venous sinus thrombosis- with right temporal lobe infarction, Dysentry, Encephalopathy, Epilepsy, Focal convulsions, Gastroenteritis- with hypoxic-ischemic encephalopathy, Gastroenteritis caused by shock, Guillain Barre, hypoxic-ischemic encephalopathy, HIV- with disseminated TB, HIV encephalopathy and focal seizures, Idiopathic intracranial hypertension and pseudotumor cerebri, epilepsy and break through seizures, Leukemia, Miliary TB- with lymphocytic interstitial pneumonitis, multilobar pneumonia, organophosphate poisoning, pigmentary retinopathy, pneumonia, Rhomencephalitis, RSV pneumonia, Salicylate poisoning, status epilepsy- with complex febrile seizure, Viral gastroenteritis (Adenovirus and Rotavirus) with encephalopathy, Viral pneumonia- with SAM and Nosocomial sepsis).

### Utility of Individual Host CSF Protein Biomarkers in the Diagnosis of TBM

Differences were assessed in the concentrations of the 67 protein biomarkers that were selected for evaluation in this study, between children diagnosed with (n=39) and without TBM (n=48). Out of the 67 biomarkers evaluated, 55 were significantly different (p<0.05) between the two groups irrespective of HIV infection status, according to the Mann Whitney U test. After correcting for multiple testing using the Bonferroni method, 48 remained significantly different between the two groups (p<0.0007) (Table 3).

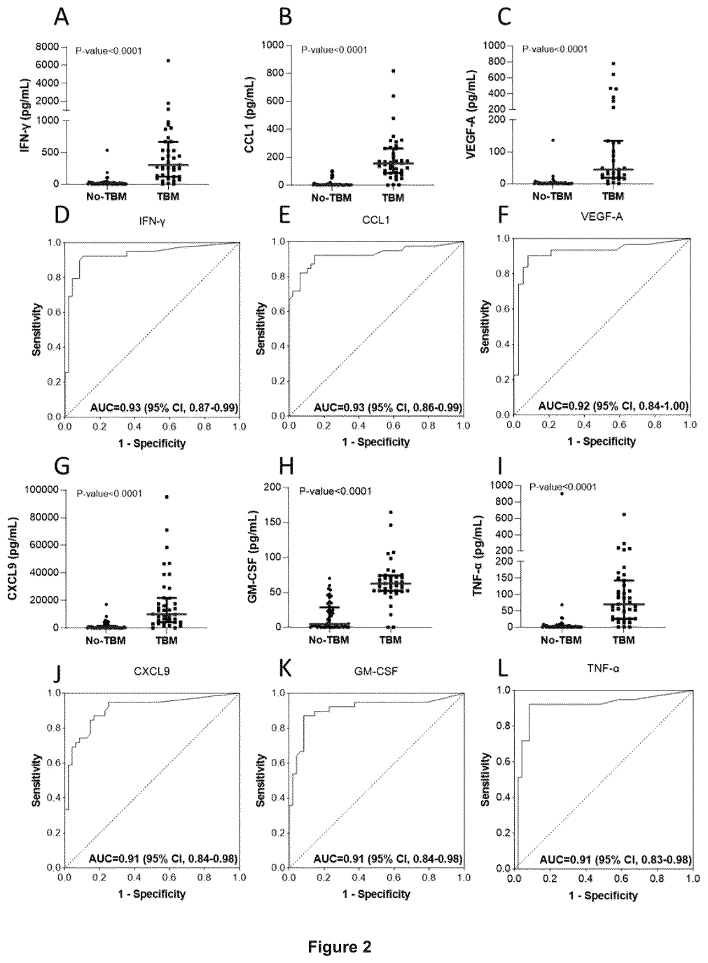

When ROC curve analysis was used to investigate the abilities of individual biomarkers to diagnose TBM irrespective of HIV status of the study participant, the area under the curve (AUC) was between 0.80 and 0.93 for 33 of the 67 proteins, namely: IFN-γ, CCL1 (I-309), VEGF-A, GM-CSF, CXCL9 (MIG), TNF-α, IL-10, MMP-8, CCL5 (RANTES), CXCL8 (IL-8), CCL18 (MIP-4), IL-1β, CXCL11 (I-TAC), S100A9, IL-18, CCL4 (MIP-1β), IL-12/23p40, CD40 ligand, Complement component (CC) 2, IL-6, PAI-1, CCL22 (MDC), MPO, CC5, IL-1RA, CC5a, MMP-1, MBL, CC4b, apolipoprotein AI, CXCL10 (IP-10), CC4, and G-CSF. Scatter plots and ROC curves for the six most accurate biomarkers (AUC≥0.90) are shown in FIG. 2.

TABLE 3
Utility of individual protein biomarkers measured in cerebrospinal fluid samples from children with suspected meningitis in the
diagnosis of TBM. Median concentrations of host biomarkers detected in CSF samples from children with and without TBM are shown (with
interquartile ranges in parenthesis). The p-values were determined by Mann-Whitney U test. The optimal cut-off values, and the associated
sensitivities and specificities were selected based on the highest Youden's index.

| | Median (IQR): | Median (IQR): | | | Optimal | Sensitivity % | Specificity % |
|---|---|---|---|---|---|---|---|
| Marker | No-TBM | TBM | P-value | AUC (95% CI) | cut-off | (95% CI) | (95% CI) |
| IFN-γ* | 6.1 (0.0-20.5) | 301.0 (118.3-669.00 | <0.0001 | 0.93 (0.87-0.99) | 42.82 | 92.3 (79.1-98.4) | 89.6 (77.3-96.5) |
| CCL1 (I-309)* | 2.4 (1.2-5.6) | 154.7 (85.6-262.4) | <0.0001 | 0.93 (0.86-0.99) | 29.215 | 92.3 (79.1-98.4) | 85.4 (72.2-93.9) |
| VEGF-A* | 1.2 (0.1-2.9) | 44.7 (18.7-134.2) | <0.0001 | 0.92 (0.84-1.00) | 8.145 | 90.3 (74.2-98.0) | 92.1 (78.6-98.3) |
| GM-CSF* | 4.7 (0.0-27.5) | 62.6 (52.3-73.9) | <0.0001 | 0.91 (0.84-0.98) | 47.64 | 87.2 (72.6-95.7) | 91.7 (80.0-97.7) |
| CXCL9 (MIG)* | 366.3 (0.0-1417.2) | 10058.7 (4246.8- | <0.0001 | 0.91 (0.84-0.98) | 2802.385 | 87.2 (72.6-95.7) | 83.3 (69.8-92.5) |
| | | 21874.6) | | | | | |
| TNF-α* | 0.7 (0.0-3.5) | 69.8 (25.5-142.0) | <0.0001 | 0.91 (0.83-0.98) | 12.77 | 92.3 (79.1-98.4) | 91.7 (80.0-97.7) |
| IL-10* | 5.6 (2.8-11.9) | 50.1 (24.2-90.3) | <0.0001 | 0.88 (0.80-0.96) | 19.645 | 87.2 (72.6-95.7) | 85.4 (72.2-93.9) |
| MMP-8* | 98.8 (25.7-455.2) | 10780.0 (4140.7- | <0.0001 | 0.88 (0.80-0.96) | 1409.905 | 87.2 (72.6-95.7) | 83.3 (69.8-92.5) |
| | | 19632.1) | | | | | |
| CCL5 (RANTES)* | 0.0 (0.0-8.1) | 43.7 (28.1-94.0) | <0.0001 | 0.88 (0.80-0.96) | 14.68 | 87.2 (72.6-95.7) | 83.3 (69.8-92.5) |
| CXCL8 (IL-8)* | 89.4 (35.5-207.9) | 799.0 (437.6-1901.0) | <0.0001 | 0.87 (0.80-0.95) | 243.78 | 87.2 (72.6-95.7) | 81.3 (67.4-91.1) |
| #CCL18 (MIP-4)* | 0.2 (0.1-1.4) | 32.8 (9.7-63.7) | <0.0001 | 0.87 (0.79-0.96) | 4.625 | 82.1 (66.5-92.5) | 91.7 (80.0-97.7) |
| IL-1β* | 0.0 (0.0-3.8) | 23.6 (11.2-52.2) | <0.0001 | 0.87 (0.79-0.95) | 7.515 | 87.2 (72.6-95.7) | 85.4 (72.2-93.9) |
| CXCL11 (I-TAC)* | 23.5 (2.2-56.0) | 226.9 (90.0-311.9) | <0.0001 | 0.87 (0.79-0.95) | 87.585 | 79.5 (63.5-90.7) | 85.4 (72.2-93.9) |
| S100A9* | 72.0 (37.8-185.5) | 3208.7 (974.2- | <0.0001 | 0.87 (0.79-0.95) | 364.425 | 84.6 (69.5-94.1) | 85.4 (72.2-93.9) |
| | | 9253.9) | | | | | |
| IL-18* | 17.2 (10.5-37.1) | 144.7 (82.1-242.8) | <0.0001 | 0.86 (0.77-0.94) | 64.49 | 84.6 (69.5-94.1) | 83.3 (69.8-92.5) |
| CCL4 (MIP-1B)* | 143.3 (85.0-241.0) | 442.0 (300.6-528.0) | <0.0001 | 0.85 (0.77-0.94) | 259.505 | 84.6 (69.5-94.1) | 81.3 (67.4-91.1) |
| IL-12/23p40* | 0.0 (0.0-200.8) | 542.3 (275.2-769.4) | <0.0001 | 0.85 (0.77-0.93) | 266.32 | 84.6 (69.5-94.1) | 79.2 (65.0-89.5) |
| CD40 Ligand* | 82.3 (0.0-248.1) | 827.9 (515.2-1195.80 | <0.0001 | 0.85 (0.76-0.94) | 404.62 | 87.2 (72.6-95.7) | 83.3 (69.8-92.5) |
| #Complement C2* | 12.8 (2.4-115.8) | 626.5 (275.1-1253.8) | <0.0001 | 0.85 (0.76-0.94) | 255.845 | 79.5 (63.5-90.7) | 89.6 (77.3-96.5) |
| IL-6* | 6.1 (2.0-36.8) | 682.2 (195.1-2760.4) | <0.0001 | 0.85 (0.76-0.94) | 110.7 | 84.6 (69.5-94.1) | 81.3(67.4-91.1) |
| PAI-1* | 505.9 (237.6-1244.9) | 6161.4 (2419.9- | <0.0001 | 0.85 (0.76-0.94) | 2280.65 | 82.1 (66.5-92.5) | 83.3 (69.8-92.5) |
| | | 15030.2) | | | | | |
| CCL22 (MDC)* | 16.7 (0.0-22.5) | 105.9 (26.7-340.3) | <0.0001 | 0.85 (0.76-0.93) | 23.765 | 82.1 (66.5-92.5) | 77.1 (62.7-88.0) |
| MPO* | 1082.7 (0.0-8082.3) | 64218.0 (45684.9- | <0.0001 | 0.85 (0.76-0.93) | 32692.63 | 84.6 (69.5-94.1) | 83.3 (69.8-92.5) |
| | | 79110.8) | | | | | |
| #Complement C5* | 9.9 (0.7-53.8) | 326.4 (122.4-731.6) | <0.0001 | 0.84 (0.75-0.93) | 66.66 | 79.5 (63.5-90.7) | 79.2 (65.0-89.5) |
| IL-1RA* | 262.7 (140.5-1304.8) | 19004.8 (5992.5- | <0.0001 | 0.84 (0.74-0.93) | 2905.285 | 89.7 (75.8-97.1) | 83.3 (69.8-92.5) |
| | | 1758000.0) | | | | | |
| #Complement | 2.5 (0.3-6.2) | 29.5 (9.5-94.6) | <0.0001 | 0.83 (0.74-0.93) | 9.165 | 76.9 (60.7-88.9) | 83.3 (69.8-92.5) |
| C5a* | | | | | | | |
| MMP-1* | 9.4 (0.0-21.5) | 91.8 (37.1-171.7) | <0.0001 | 0.82 (0.73-0.92) | 31.185 | 82.1 (66.5-92.5) | 81.3 (67.4-91.1) |
| #MBL* | 0.3 (0.0-1.8) | 9.0 (1.6-27.7) | <0.0001 | 0.82 (0.73-0.91) | 1.475 | 79.5 (63.5-90.7) | 75.0 (60.4-86.4) |
| #Complement | 61.9 (29.9-139.1) | 228.7 (139.3-310.2) | <0.0001 | 0.82 (0.73-0.91) | 105.435 | 87.2 (72.6-95.7) | 68.8 (53.7-81.3) |
| C4b* | | | | | | | |
| #Apo-Al* | 0.0 (0.0-60.0) | 1070.2 (439.3- | <0.0001 | 0.81 (0.72-0.91) | 221.625 | 82.1 (66.5-92.5) | 79.2 (65.0-89.5) |
| | | 2737.3) | | | | | |
| CXCL10 (IP-10)* | 55.6 (14.9-827.4) | 1984.4 (1556.0- | <0.0001 | 0.81 (0.71-0.91) | 491.51 | 89.7 (75.8-97.1) | 75.0 (60.4-86.4) |
| | | 2200.0) | | | | | |
| #Complement C4* | 612.4 (271.6-935.9) | 1735.2 (795.1- | <0.0001 | 0.80 (0.70-0.90) | 667.59 | 89.7 (75.8-97.1) | 64.6 (49.5-77.8) |
| | | 2835.4) | | | | | |
| G-CSF* | 47.7 (12.5-105.6) | 279.9 (123.4-620.7) | <0.0001 | 0.80 (0.70-0.90) | 113.665 | 79.5 (63.5-90.7) | 79.2 (65.0-89.5) |
| #sP-Selectin* | 0.3 (0.1-0.6) | 0.9 (0.5-2.3) | <0.0001 | 0.79 (0.70-0.89) | 0.515 | 75.7 (58.8- 88.2) | 72.9 (58.2-84.7) |
| #Complement | 83.3 (53.1-194.0) | 345.4 (149.5-511.5) | <0.0001 | 0.79 (0.69-0.89) | 125.185 | 84.6 (69.5-94.1) | 66.7 (51.6-79.6) |
| Factor I* | | | | | | | |
| #Lipocalin- | 1.5 (0.8-5.3) | 10.2 (4.1-14.5) | <0.0001 | 0.79 (0.69-0.89) | 3.435 | 87.2 (72.6-95.7) | 68.8 (53.7-81.3) |
| 2/NGAL* | | | | | | | |
| IL-1α* | 0.0 (0.0-3.6) | 7.9 (4.1-10.4) | <0.0001 | 0.78 (0.68-0.88) | 3.835 | 79.5 (63.5-90.7) | 77.1 (62.7-88.0) |
| ICAM-1* | 526.8 (197.2-1357.7) | 2424.6 (1165.9- | <0.0001 | 0.77 (0.66-0.87) | 1628.525 | 69.2 (52.4-83.0) | 81.3 (67.4-91.1) |
| | | 3490.7) | | | | | |
| IL-4* | 47.8 (17.4-80.9) | 89.8 (65.8-106.1) | <0.0001 | 0.77 (0.66-0.87) | 62.205 | 84.6 (69.5-94.1) | 66.7 (51.6-79.6) |
| PDGF AB/BB* | 4.3 (2.4-7.3) | 12.3 (6.9-40.2) | <0.0001 | 0.77 (0.66-0.87) | 6.75 | 79.5 (63.5-90.7) | 70.8 (55.9-83.0) |
| #Complement | 707.5 (493.4-1638.6) | 3486.6 (1725.2- | <0.0001 | 0.76 (0.65-0.87) | 2085.185 | 74.4 (57.9-87.0) | 79.2 (65.0-89.5) |
| factor H* | | 6775.0) | | | | | |
| MMP-9* | 25.7 (10.1-204.5) | 782.3 (98.5-2120.1) | <0.0001 | 0.76 (0.61-0.91) | 69.29 | 82.4 (56.6-96.2) | 71.4 (51.3-86.8) |
| #Complement C3* | 0.0 (0.0-191.0) | 486.9 (116.4-2102.5) | <0.0001 | 0.75 (0.65-0.86) | 112.08 | 76.9 (60.7-88.9) | 72.9 (58.2-84.7) |
| #Complement | 12.1 (7.5-21.0) | 32.9 (15.3-60.2) | <0.0001 | 0.75 (0.64-0.86) | 25.59 | 66.7 (49.8-80.9) | 83.3 (69.8-92.5) |
| Factor D* | | | | | | | |
| #SAP* | 7.4 (3.2-22.7) | 37.1 (15.5-125.5) | <0.0001 | 0.75 (0.64-0.85) | 21 | 71.8 (55.1-85.0) | 75.0 (60.4-86.4) |
| #α1-Antitrypsin* | 137.3 (42.9-610.0) | 750.0 (196.3-7172.5) | 0.0003 | 0.73 (0.62-0.84) | 432.34 | 66.7 (49.8-80.9) | 72.9 (58.2-84.7) |
| IL-17* | 0.0 (0.0-0.0) | 4.1 (0.0-15.1) | 0.0001 | 0.73 (0.62-0.84) | 0.85 | 71.8 (55.1-85.0) | 79.2 (65.0-89.5) |
| VCAM-1* | 42494.6 (20713.6- | 113138.4 (55541.3- | 0.0005 | 0.72 (0.61-0.83) | 70698.47 | 69.2 (52.4-83.0) | 66.7 (51.6-79.6) |
| | 93918.5) | 170498.8) | | | | | |
| CD45 (NCAM) | 46047.4 (32721.7- | 30009.0 (22473.2- | 0.0061 | 0.67 (0.56-0.79) | 37585.86 | 64.6 (49.5-77.8) | 66.7 (49.8-80.9) |
| | 52709.7) | 42955.9) | | | | | |
| #Apo-CIII | 63.5 (18.3-243.4) | 266.0 (50.8-491.1) | 0.0068 | 0.67 (0.55-0.78) | 85.305 | 71.8 (55.1-85.0) | 60.4 (45.3-74.2) |
| CCL3 (MIP-1α) | 263.9 (181.1-391.1) | 355.8 (262.5-604.1) | 0.0101 | 0.66 (0.55-0.78) | 284.66 | 69.2 (52.4-83.0) | 60.4 (45.3-74.2) |
| PDGF-AA | 2.7 (1.2-5.2) | 6.2 (2.0-14.1) | 0.0172 | 0.65 (0.53-0.77) | 3.55 | 61.5 (44.6-76.6) | 64.6 (49.5-77.8) |
| #SAA | 14.0 (4.1-71.0) | 103.6 (4.8-202700.0) | 0.0161 | 0.65 (0.53-0.77) | 89.63 | 60.5 (43.4-76.0) | 81.3 (67.4-91.1) |
| #D-DIMER | 106.3 (21.4-279.5) | 550.5 (46.5-97700.0) | 0.0253 | 0.64 (0.52-0.76) | 232.155 | 56.4 (39.6-72.2) | 72.9 (58.2-84.7) |
| Aβ42 | 347.7 (99.6-906.6) | 147.2 (55.3-456.2) | 0.0386 | 0.63 (0.51-0.75) | 428.615 | 47.9 (33.3-62.8) | 74.4 (57.9-87.0) |
| #CRP | 408.2 (43.9-2647.5) | 1882.5 (456.4- | 0.0692 | 0.61 (0.49-0.73) | 819.895 | 71.8 (55.1-85.0) | 56.3 (41.2-70.5) |
| | | 833100.0) | | | | | |
| CCL11 (Eotaxin) | 0.0 (0.0-27.1) | 0.0 (0.0-74.0) | 0.0526 | 0.61 (0.49-0.73) | 23.905 | 48.7 (32.4-65.2) | 72.9 (58.2-84.7) |
| FAP | 230.4 (69.4-473.7) | 346.5 (143.1-925.1) | 0.2020 | 0.61 (0.44-0.79) | 454.475 | 47.1 (23.0-72.2) | 75.0 (55.1-89.3) |
| Transthyretin | 5.4 (3.9-8.3) | 6.9 (4.3-11.7) | 0.1395 | 0.60 (0.47-0.72) | 5.712 | 67.6 (49.5-82.6) | 56.3 (41.2-70.5) |
| IL-13 | 0.0 (0.0-11.0) | 8.4 (0.0-35.3) | 0.1224 | 0.59 (0.47-0.72) | 13.975 | 48.6 (31.9-65.6) | 80.9 (66.7-90.9) |
| #GDF-15 | 0.1 (0.0-0.2) | 0.1 (0.1-0.3) | 0.1486 | 0.59 (0.47-0.71) | 0.085 | 66.7 (49.8-80.9) | 56.3 (41.2-70.5) |
| CCL2 (MCP-1) | 895.7 (560.8-1739.0) | 540.6 (367.1-1169.9) | 0.1591 | 0.59 (0.47-0.71) | 549.54 | 77.1 (62.7-88.0) | 51.3 (34.8-67.6) |
| sRAGE | 3.0 (0.0-4.2) | 0.0 (0.0-4.2) | 0.2342 | 0.57 (0.45-0.69) | 1.5 | 58.3 (43.2-72.4) | 56.4 (39.6-72.2) |
| TGF-α | 6.7 (0.0-10.6) | 0.0 (0.0-9.1) | 0.2357 | 0.57 (0.45-0.69) | 2.78 | 60.4 (45.3-74.2) | 56.4 (39.6-72.2) |
| Ferritin | 252.3 (107.7-363.5) | 366.7 (103.2-934.9) | 0.4397 | 0.57 (0.39-0.75) | 361.93 | 52.9 (27.8-77.0) | 75.0 (55.1-89.3) |
| #PEDF | 730.6 (293.4-923.2) | 572.0 (349.6-1160.8) | 0.3611 | 0.56 (0.43-0.68) | 681.955 | 59.0 (25.6-57.9) | 54.2 (31.4-60.8) |
| Procalcitonin | 184.5 (123.2-296.4) | 186.0 (127.7-283.3) | 0.8645 | 0.51 (0.39-0.63) | 172.28 | 45.8 (39.2- 68.6) | 61.5 (23.4-55.4) |

*Biomarkers that remained significantly different after correction for multiple testing using Bonferroni method (p < 0.0007).
#Concentrations reported in ng/ml, all others concentrations are reported in pg/mL.
VEGF-A data obtained from LXSAHM-01 was used for analysis due to insufficient values when using HCYTA-60K-03.

### Identification of CSF Protein Biosignatures for the Diagnosis of TBM

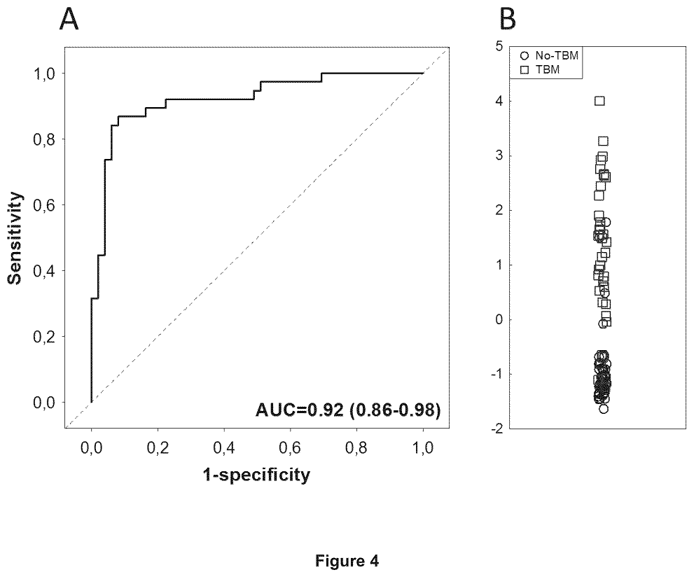

The data obtained for all the 67 biomarkers investigated was fitted into GDA models irrespective of HIV infection status, and without any constraints on the identity and/or the number of biomarkers in the model. Optimal diagnosis of TBM was shown to be achieved with a combination of up to 5 biomarkers. However, the most accurate biosignature identified was a 4-marker model composed of CC4b, CC4, procalcitonin, and CCL1, which diagnosed TBM with an AUC of 0.97 (95% CI, 0.93-1.00), sensitivity of 92.3% (24/26) (95% CI, 74.9-99.1) and specificity of 100.0% (38/38) (95% CI, 92.4-100%). After leave-one-out cross-validation, the sensitivity of the signature was 84.6% (22/26) (95% CI, 65.1-95.6) and specificity was 94.7% (36/38) (95% CI, 82.3-99.4). At the WHO TTP recommended sensitivity threshold of ≥80%, the specificity of the 4-marker model was 100%, meeting the optimal requirements. An alternative 4-marker signature comprising apolipoprotein-CIII, CC4, RANTES and TNF-α also showed promise in the diagnosis of TBM, with AUC of 0.92 (95% CI, 0.86-0.98) (Table 4, FIG. 4).

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

TABLE 4
Sumarry: Diagnostic accuracy of identified biosignatures.

| | | | | | | | | At the | |
|---|---|---|---|---|---|---|---|---|---|
| | Diagnostic accuracy of the | | | Diagnostic accuracy after leave-one-out cross- | | | | threshold of | |
| | biosignature | | | validation | | | | ≥80% | |
| | AUC | Sens % | Spec % | Sens % | Spec % | PPV % | NPV % | sensitivity | |
| Biosignature | (95% CI) | (95% CI) | (95% CI) | (95% CI) | (95% CI) | (95% CI) | (95% CI) | Sens | Spec |
| CC4b + CC4 + | 0.98 | 89.5 | 100.0 | 81.6 | 100.0 | 100.0 | 87.5 | 89% | 100% |
| procalcitonin + | (0.94-1.00) | (75.2-97.1) | (94.1-100.0) | (65.7-92.3) | (94.1-100.0) | (92.7-100.0) | (78.2-93.2) | | |
| CCL1 | | | | | | | | | |
| Apo-CIII + CC4 + | 0.92 | 78.9 | 93.9 | 78.9 | 91.8 | 88.2 | 84.9 | 84% | 94% |
| RANTES + TNF-α | (0.86-0.98) | (62.7-90.4) | (83.1-98.7) | (62.7-90.4) | (80.4-97.7) | (74.3-95.1) | (75.1-91.3) | | |

^{#}The accuracies of the biosignatures were benchmarked against the World Health Organization target product profiles (WHO TPP) for a rapid biomarker-based test for all forms of extrapulmonary TB in adults (sensitivity ≥ 80% and specificity ≥ 98%).
Sens = sensitivity,
spec = specificity,
PPV = positive predictive value,
NPV = negative predictive value.

1-10. (canceled)

11. A device for diagnosing TB, the device comprising:
a means for receiving a biological sample from a subject suspected of having TB;
capture agents for binding CC4 and at least one other biomarker selected from the group consisting of CC4b, procalcitonin, CCL1, apolipoprotein-CIII, RANTES and TNF-α; and
at least one indicator to indicate to a user of the device when the capture agents bind to the biomarkers.

12. The device as claimed in claim 11, which comprises capture agents for binding CC4, CC4b, procalcitonin and CCL1.

13. The device as claimed in claim 11, which comprises capture agents for binding CC4, apolipoprotein-CIII, RANTES and TNF-α.

14. The device as claimed in claim 11, wherein the capture agents are selected from the group consisting of antibodies, affibodies, ankyrin repeat proteins, armadillo repeat proteins, nucleic acid aptamers, peptides, carbohydrate ligands, synthetic ligands and synthetic polymers.

15. The device as claimed in claim 14, wherein the capture agents are antibodies.

16. The device as claimed in claim 11, wherein the device includes measuring means for measuring the levels of the detected biomarkers.

17. A kit for diagnosing TB, the kit comprising one or more of the following:
capture agents for binding CC4 and at least one other biomarker selected from the group consisting of CC4b, procalcitonin, CCL1, apolipoprotein-CIII, RANTES and TNF-α;
means for obtaining or receiving a biological sample from a subject;
a device for diagnosing TB; and/or
instructions, in electronic or paper form, for performing the method as described above.

18. The kit as claimed in claim 17, which comprises capture agents for binding CC4, CC4b, procalcitonin and CCL1.

19. The kit as claimed in claim 17, which comprises capture agents for binding CC4, apolipoprotein-CIII, RANTES and TNF-α.

20. A method of diagnosing a human subject as having TB and treating the subject, the method comprising the steps of:
testing a biological sample from a subject suspected of having TB for the presence of CC4 and at least one other biomarker selected from the group consisting of CC4b, procalcitonin, CCL1, apolipoprotein-CIII, RANTES and TNF-α;
determining whether the subject has TB based on the detection of the biomarkers in the sample; and
administering an effective amount of TB treatment to the subject.

21-24. (canceled)

25. The method as claimed in claim 20, wherein the sample is tested for the presence of CC4 and two other biomarkers selected from the group consisting of CC4b, procalcitonin, CCL1, apolipoprotein-CIII, RANTES and TNF-α.

26. The method as claimed in claim 20, wherein the sample is tested for the presence of CC4 and three other biomarkers selected from the group consisting of CC4b, procalcitonin, CCL1, apolipoprotein-CIII, RANTES and TNF-α.

27. The method as claimed in claim 20, wherein the TB is TB meningitis or spinal TB.

28. The method as claimed in claim 20, wherein the sample is a cerebrospinal fluid (CSF), saliva, sputum, blood, urine, or pleural or pericardial effusion sample.

29. The method as claimed in claim 28, wherein the sample is a cerebrospinal fluid (CSF) sample.

30. The method as claimed in claim 20, wherein the sample is tested for the presence of CC4, CC4b, procalcitonin and CCL1.

31. The method as claimed in claim 20, wherein the sample is tested for the presence of CC4, apolipoprotein-CIII, RANTES and TNF-α.

32. The method as claimed in claim 20, wherein the biological sample is contacted with capture agents which bind to the biomarkers and binding of the capture agents to the biomarkers is detected.

33. The method as claimed in claim 20, wherein detection of two or more of the biomarkers in the sample or a measured signal which equates to a level of biomarker in the sample which is higher than a threshold level of the same biomarker is an indicator of TB.

34. The method as claimed in claim 20, wherein the TB is spinal TB.
